SEQUENCE LISTING (2 sequences)

SEQ ID NO: 1 - DNA, 31 bp, Artificial
  sense linker oligonucleotide
ctagtatcgattgttaattaagggcgtggcc

SEQ ID NO: 2 - DNA, 31 bp, Artificial
  antisense linker oligonucleotide
ttaaggccacgcccttaattaacaatcgata

(19) United States
(12) Patent Application Publication
(10) Pub. No.: US 2007/0105794 A1
(43) Pub. Date: May 10, 2007

(54) Immunotherapy

(76) Inventors: Kai S. Lipinski, Staffordshire (GB); Hakim Djeha, Cheshire (GB)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/531,002

(22) PCT Filed: Oct. 10, 2003
(86) PCT No.: PCT/GB03/04388

§ 371 Date: Apr. 11, 2006

(30) Foreign Application Priority Data
Oct. 14, 2002 (GB) 0223696.6

(51) Int. Cl. A61K 48/00; C07H 21/04

(52) U.S. Cl. 514/44; 536/23.2

(57) ABSTRACT

The invention provides an improved method of inducing an immune response against targeted cells. Using gene therapy to express both a toxin or prodrug converting enzyme as a means of killing a targeted cell type and also stress response protein (in particular, a heat shock protein) enhances the subsequent immune response directed against such cells. The method is particularly applicable to inducing an immune response against cancer cells. Also provided are polynucleotides, products and vectors for use in such a method.

### FIELD OF THE INVENTION

This invention relates to the field of immunotherapy, in particular to the enhancement of anti-tumour immune responses.

### BACKGROUND TO THE INVENTION

One approach to treating cancer is to introduce a gene into the tumour cells that encodes an enzyme capable of converting a prodrug of relatively low toxicity into a potent cytotoxic drug. Systemic administration of the prodrug is then tolerated since it is only converted into the toxic derivative locally, in the tumour, by cells expressing the prodrug-converting enzyme. This approach is known as gene-directed enzyme prodrug therapy (GDEPT), or when the gene is delivered by means of a recombinant viral vector, virus-directed prodrug therapy (VDEPT) (McNeish et al, 1997).

Examples of prodrugs and prodrug-converting enzymes used in this way include ganciclovir and HSV thymidine kinase, 5-fluorocytosine and cytosine deaminase, cyclophosphamide or paracetamol and cytochrome P450, and, of particular relevance to this invention, the aziridinyl prodrug CB1954 (5-(aziridin-1-yl)-2,4-dinitrobenzamide) and nitroreductase (Knox et a/1988).

Following the observation that the Walker rat carcinoma cell line was particularly sensitive to CB1954, it was shown that this was due to the expression of the rat nitroreductase DT diaphorase. However, since CB1954 is a poor substrate for the human form of this enzyme, human tumour cells are far less sensitive to CB1954. GDEPT was conceived as a way of introducing a suitable nitroreductase, preferably with greater activity against CB1954, in order to sensitise targeted cells. The Escherichia coli nitroreductase (EC1.6.99.7, alternatively known as the oxygen-insensitive NAD(P)H nitroreductase or dihydropteridine reductase, and often abbreviated to NTR) encoded by the NFSB gene (alternatively known as NFNB, NFSI, or DPRA) has been widely used for this purpose (Reviewed in Grove et al, 1999). The NFSB-encoded nitroreductase (NTR) is a homodimer that binds two flavin mononucleotide (FMN) cofactor molecules.

Using NADH or NADPH as an electron donor, and bound FMN as a reduced intermediate, NTR reduces one or other of the two nitro-groups of CB 1954 to give either the highly toxic 4-hydroxylamine derivative or the relatively non-toxic 2-hydroxylamine. Within cells, 5-(aziridin-1-yl)-4-hydroxylamino-2-nitrobenzamide, probably via a further toxic metabolite, becomes very genotoxic (Knox et al, 1991). The exact nature of the lesion caused is unclear, but is unlike that caused by other agents. A particularly high rate of inter-strand cross-linking occurs and the lesions seem to be poorly repaired, with the result that CB 1954 is an exceptionally affective anti-tumour agent (Friedlos et al, 1992). This system has been show to be effective in producing useful anti-tumoral responses in a number of model systems, including human xenografted tumours in nude mice (Djeha et al, 2000)

That said, attempts have been made to enhance the tumoricidal effects of such enzyme-prodrug strategies by combining them with immunotherapeutic approaches. The underlying rationale is to try to generate an anti-tumour immune response to tumour-specific antigens in the cells killed by the cytotoxic agent, in the hope that residual cells at the tumour site, and at metastases, will be killed by (predominantly) a cytotoxic T cell response. In order to help stimulate such a response, gene therapy using a number of immunostimulatory molecules has been attempted in combination with GDEPT/VDEPT. One of the better candidates appears to be GM-CSF, which has been used in combination with thymidine kinase (Jones et al) and with cytosine deaminase (Cao et al). Other candidates tried in combination with one or both include IL-2, II-6, B7-1 (Felzman et al), IFN-γ (Santodonato et al and MCP-1 (Sakai et al).

However, in general, the results have been variable and have not led to reliable improvements in therapy. There remains a clear need for improvements in the immunotherapy of cancer.

In response to cellular stress, a subset of genes are induced resulting in the expression of stress response proteins. These proteins have diverse functions and include intracellular messengers and transcription factors, such as NFκB and high mobility group B1 (HMGB1, Bustin, 2002), and cytokines such as IL-1β, IL-1α, IL-6, IL-8, TNF-α, GM-CSF, IL-12 and IL-15. Amongst the most important, however are the group of proteins known as heat shock proteins.

Heat shock proteins are ubiquitous intracellular proteins, highly conserved through evolution and known to be involved in basic cellular processes such as folding, unfolding and degradation of proteins, and assembly of multi-subunit protein complexes. Their expression is sharply upregulated by cellular stress (heat, toxins, starvation, hypoxia) and they have a role in repairing and ameliorating protein damage caused during periods of cellular stress (reviewed in Parsell and Lindquist, 1993). Expression of heat shock proteins is controlled by a number of upstream factors that bind to elements in heat shock protein promoters. These include HSF-1 (heat shock factor-1, Baler et al., 1993), HSF-2 (Mathew et al, 2001), and HSF-3 (Nakai and Morimoto, 1993), and the interferon response factors, including IRF-1 and 2 (Taniguchi et al, 2001, Mamane et al, 1999).

More recently, it has become clear that they also play a role in the processing and presentation of peptides derived from intracellular proteins, and the delivery of these to antigen presenting cells. In particular, the heat shock proteins Hsp70, Hsp90 and calreticulin bind self peptides with extremely high affinity, and on being released from the cell by lysis deliver them to antigen presenting cells expressing the Hsp receptor CD91 (Basu et al, 2001). The cellular stress before lysis increases the levels of heat shock proteins and so increases this effect. It has been proposed that stimulation of the immune response against intracellular antigens is related to necrotic, rather than apoptotic, or programmed, cell death and that this is, at least partly, controlled at the level of antigen presenting cells (Matzinger, 1994). As applied specifically to anti-tumour responses, it has been shown that non-apoptotic (ie necrotic) cell death of tumour cells induces higher levels of Hsp70 and is associated with increased immunogenicity, release of inflammatory cell contents, increased secretion of pro-inflammatory Th1-type cytokines and macrophage activation (Gough et al., 2001). Apoptotic cell death, on the other hand, in which intracellular macromolecules are degraded before being exposed to the extracellular environment, and Hsp70, Hsp90 and calreticulin are not released in significant amounts (Basu et al, 2000) in general fails to initiate inflammatory and immune responses. Necrotic, but not apoptotic, cell lysates cause maturation of dendritic cells, the key antigen presenting cells for activating naïve T cells in the initiation of an immune response (Galucci et al, 1999; Sauter et al., 2000. Hsp70 itself has been shown to be a maturation factor for dendritic cells (Kuppner, et al, 2001; Gastpar et al., European Patent Application EP1209226). In addition, apoptotic cells may deliver specific anti-inflammatory and immunosuppressive signals directly to antigen presenting cells, for example through the phosphatidylserine receptor (Fadok et al, 2000)

### SUMMARY OF THE INVENTION

The inventors found that the use of a nitroreductase/CB1954 enzyme/prodrug system, in addition to the killing of targeted tumour cells, and of bystander cells exposed to the toxic products of activated CB1954 released from lysed cells, also produced a significant protective effect against later challenge with tumour cells of the same type. Since earlier work suggested that such killing was through apoptotic mechanisms (Djeha et al, 2000) and apoptotic cell death was thought to produce a relatively poor immune response, this was unexpected. In an effort to further enhance the anti-tumour effect they constructed adenoviral gene therapy vectors comprising coding polynucleotides capable of expressing both a nitroreductase and Hsp70. The rationale is that tumour cells targeted and infected by the vector and killed by administration of CB1954, then act to prime an anti-tumour immune response triggered by the death of the tumour cells and enhanced by overexpression of Hsp70. This, in turn, leads to more efficient delivery of intracellular peptides bound by Hsp70 to CD91-expressing antigen presenting cells. Some of the peptides so delivered contain tumour-specific, ‘non-self’ epitopes resulting from somatic mutation within the tumour cells and so representation of these in the context of (predominantly) MHC Class I molecules leads to initiation of a CD8⁺ cytotoxic T cell anti-tumour response. However, a proportion of Hsp-delivered peptides enter the MHC Class II presentation pathway and so result in increased CD4⁺ helper T cell responses. Both arms of the cellular response are important in anti-tumour immunity.

Whether the success of this approach in generating anti-tumour immune responses is because the GDEPT-mediated killing is necrotic in at least a proportion of cells, whether the primary mechanism of killing is apoptotic but a significant level of secondary necrosis occurs, or whether the anti-inflammatory and non-immunogenic nature of apoptotic cell death may be overcome to some extent by overexpression of heat shock factors, is not clear. However, the inventors have demonstrated that in a mouse tumour model, injection of a primary tumour with vector delivering NTR genes and subsequent systemic administration of CB1954 not only kills the tumour, but gives a degree of protection against subsequent challenge with tumour cells of the same type. They have further demonstrated that this protective effect is greatly enhanced by the co-administration, conveniently in the same gene therapy vector, of a gene directing expression of Hsp70.

This invention is not limited to the use of nitroreductase as the prodrug-converting enzyme, nor to methods of cell killing relying on prodrug conversion. In principle, any method of cell killing, including polynucleotides encoding molecules capable of cell killing, may be used in combination with any molecule with a heat shock protein-like function in acting as an endogenous adjuvant and boosting an anti-tumour response. Among the toxins that could be encoded and used in this way are ricin, abrin, diphtheria toxin and botulinum toxin. Among the alternative prodrug-converting enzymes suitable is the cytochrome P450/acetaminophen enzyme/prodrug combination. It is known that such system is useful as a means of killing tumour cells (International Patent Application WO 00/40272), and there is some evidence that the cell killing that results from the activation of acetaminophen to the toxic metabolite N-acetylbenzoquinoneimine (NABQI) is apoptotic (Bae et al, 2001; Boulares et al, 2002). Nevertheless, the applicants have found that coexpression of cytochrome P450 and hsp70, in the presence of therapeutic levels of acetaminophen, give significantly improved anti-tumour responses over those obtained with cytochrome P450 alone.

Indeed, it is clear that the disclosed approach of delivering vectors providing expression of heat shock proteins may be used to enhance immune responses against cells, such as (but not limited to) tumour cells, killed by any of a variety of methods. Thus DNA delivery of heat shock proteins as adjunct immunotherapy combined with chemotherapy or radiotherapy is likely to be beneficial.

Among the molecules with suitable adjuvant properties that may be used are Hsp70, Hsp90, Hsp110, calreticulin, gp96, grp170, Hsp27, Hsc70, Mycobacterium Hsp65, Legionella pneumophila Hsp60, Escherichia coli GroEL and GroES.

Further, it is clear from data disclosed here that the use of a GDEPT-based approach that results in necrotic cell death (such as the NTR/CB1954 combination) is significantly immunogenic even without the extra beneficial effects of upregulated Hsp expression provided by the associated Hsp transgene.

The vector may be any vector capable of transferring DNA to a cell. Preferably, the vector is an integrating vector or an episomal vector.

Preferred integrating vectors include recombinant retroviral vectors. A recombinant retroviral vector will include DNA of at least a portion of a retroviral genome which portion is capable of infecting the target cells. The term “infection” is used to mean the process by which a virus transfers genetic material to its host or target cell. Preferably, the retrovirus used in the construction of a vector of the invention is also rendered replication-defective to remove the effect of viral replication of the target cells. In such cases, the replication-defective viral genome can be packaged by a helper virus in accordance with conventional techniques. Generally, any retrovirus meeting the above criteria of infectiousness and capability of functional gene transfer can be employed in the practice of the invention.

Suitable retroviral vectors include but are not limited to pLJ, pZip, pWe and pEM, well known to those of skill in the art. Suitable packaging virus lines for replication-defective retroviruses include, for example, ΨCrip, ΨCre, Ψ2 and ΨAm. Particularly suitable retroviral vectors are lentiviral vectors, especially HIV, SIV and FIV.

Other vectors useful in the present invention include adenovirus, adeno-associated virus, SV40 virus, vaccinia virus, HSV and poxvirus vectors. A preferred vector is the adenovirus. Adenovirus vectors are well known to those skilled in the art and have been used to deliver genes to numerous cell types, including airway epithelium, skeletal muscle, liver, brain and skin (Hitt et al, 1997; Anderson, 1998) and to tumours (Mountain, 2000).

A further preferred vector is the adeno-associated (AAV) vector. AAV vectors are well known to those skilled in the art and have been used to stably transduce human T-lymphocytes, fibroblasts, nasal polyp, skeletal muscle, brain, erythroid and haematopoietic stem cells for gene therapy applications (Philip et al, 1994; Russell et al, 1994; Flotte et al., 1993; Walsh et al., 1994; Miller et al., 1994; Emerson, 1996). International Patent Application WO 91/18088 describes specific MV based vectors.

Preferred episomal vectors include transient non-replicating episomal vectors and self-replicating episomal vectors with functions derived from viral origins of replication such as those from EBV, human papovavirus (BK) and BPV-1.

Such integrating and episomal vectors are well known to those skilled in the art and are fully described in the body of literature well known to those skilled in the art. In particular, suitable episomal vectors are described in WO98/07876.

Mammalian artificial chromosomes can also be used as vectors in the present invention. The use of mammalian artificial chromosomes is discussed by Calos (1996).

In a preferred embodiment, the vector of the present invention is a plasmid. The plasmid may be is a non-replicating, non-integrating plasmid.

The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides, and may be chemically modified by such means as methylation or the inclusion of protecting groups or cap- or tail structures.

A non-replicating, non-integrating plasmid is a nucleic acid which when transfected into a host cell does not replicate and does not specifically integrate into the host cell's genome (i.e. does not integrate at high frequencies and does not integrate at specific sites).

Replicating plasmids can be identified using standard assays including the standard replication assay of Ustav et al (1991).

The present invention also provides a host cell transfected with the vector of the present invention. The host cell may be any mammalian cell. Preferably the host cell is a rodent or mammalian cell.

Numerous techniques are known and are useful according to the invention for delivering the vectors described herein to cells, including the use of nucleic acid condensing agents, electroporation, complexing with asbestos, polybrene, DEAE cellulose, Dextran, liposomes, cationic liposomes, lipopolyamines, polyornithine, particle bombardment and direct microinjection (reviewed by Kucherlapati and Skoultchi (1984); Keown et al (1990).

A vector of the invention may be delivered to a host cell non-specifically or specifically (i.e., to a designated subset of host cells) via a viral or non-viral means of delivery. Preferred delivery methods of viral origin include viral particle-producing packaging cell lines as transfection recipients for the vector of the present invention into which viral packaging signals have been engineered, such as those of adenovirus, herpes viruses and papovaviruses. Preferred non-viral based gene delivery means and methods may also be used in the invention and include direct naked nucleic acid injection, nucleic acid condensing peptides and non-peptides, cationic liposomes and encapsulation in liposomes.

The direct delivery of vector into tissue has been described and some short-term gene expression has been achieved. Direct delivery of vector into muscle (Wolff et al, 1990), thyroid (Sikes et al, 1994), melanoma (Vile et al, 1993), skin (Hengge et al (1995), liver (Hickman et al (1994) and after exposure of airway epithelium (Meyer et al., 1995) is clearly described in the prior art.

Various peptides derived from the amino acid sequences of viral envelope proteins have been used in gene transfer when co-administered with polylysine DNA complexes (Plank et al, 1994; Trubetskoy et al, 1992; WO 91/17773; WO 92/19287; and Mack et al., 1994) suggesting that co-condensation of polylysine conjugates with cationic lipids can lead to improvement in gene transfer efficiency. International Patent Application WO 95/02698 discloses the use of viral components to attempt to increase the efficiency of cationic lipid gene transfer.

Nucleic acid condensing agents useful in the invention include spermine, spermine derivatives, histones, cationic peptides, cationic non-peptides such as polyethyleneimine (PEI) and polylysine. ‘Spermine derivatives’ refers to analogues and derivatives of spermine and include compounds as set forth in International Patent Application WO 93/18759 (published Sep. 30, 1993).

Disulphide bonds have been used to link the peptidic components of a delivery vehicle (Cotten et al., 1992). See also Trubetskoy et al. (supra).

Delivery vehicles for delivery of DNA constructs to cells are known in the art and include DNA/polycation complexes which are specific for a cell surface receptor, as described in, for example, Wu and Wu (1988), Wilson et al (1992) and U.S. Pat. No. 5,166,320.

Delivery of a vector according to the invention is contemplated using nucleic acid condensing peptides. Nucleic acid condensing peptides, which are particularly useful for condensing the vector and delivering the vector to a cell, are described in International Patent Application WO 96/41606. Functional groups may be bound to peptides useful for delivery of a vector according to the invention, as described in WO 96/41606. These functional groups may include a ligand that targets a specific cell-type such as a monoclonal antibody, insulin, transferrin, asialoglycoprotein, or a sugar. The ligand thus may target cells in a non-specific manner or in a specific manner that is restricted with respect to cell type.

The functional groups also may comprise a lipid, such as palmitoyl, oleyl, or stearoyl; a neutral hydrophilic polymer such as polyethylene glycol (PEG), or polyvinylpyrrolidine (PVP); a fusogenic peptide such as the HA peptide of influenza virus; or a recombinase or an integrase. The functional group also may comprise an intracellular trafficking protein such as a nuclear localisation sequence (NLS), an endosome escape signal such as a membrane disruptive peptide, or a signal directing a protein directly to the cytoplasm.

Accordingly, the invention provides:

A product comprising a polynucleotide sequence encoding a toxin or prodrug-converting enzyme and a polynucleotide sequence encoding a stress response protein or an inducer of stress response protein expression. Such stress response proteins include NFκB, high mobility group B1 protein HMGB1, cytokines such as IL-1β, IL-1α, IL-6, IL-8, TNF-α, GM-CSF, IL-12 and IL-15, and heat shock proteins such as Hsp70, Hsp90, Hsp110, calreticulin, gp96, grp170, Hsp27, Hsc70, Mycobacterium Hsp65, Legionella pneumophila Hsp60, Escheichia coli GroEL and GroES. Inducers of stress protein expression include HSF-1, HSF-2, HSF-3, IRF-1 and IRF-2.

In one preferred embodiment, it is preferable that the toxin or prodrug-converting enzyme is capable of killing cells neurotically. By the terms ‘necrotically’ or ‘necrotic cell death’ are included all forms of cell death that are not programmed, or apoptotic.

In a second preferred embodiment, such a product is for use in enhancing an immune response, more preferably an anti-tumour immune response.

In one alternative embodiment, the polynucleotide sequence encoding a toxin or prodrug-converting enzyme and the polynucleotide sequence encoding a stress response protein or an inducer of stress response protein expression are both components of a single polynucleotide molecule. In a further alternative, such sequences are on separate polynucleotides that may be administered concurrently or consecutively.

Preferably, the toxin or prodrug-converting enzyme is a nitroreductase capable of activating the prodrug CB1954. Alternatively, it is a cytochrome P450 enzyme, preferably of mammalian origin, more preferably of human origin. Most preferably it is human CYP1A2. Alternatively, it is human CYP2E1 or CYP3A4.

In an alternative preferred embodiment, the encoded enzyme is a rodent cytochrome P450, preferably from mouse. Most preferably, it is mouse CYP1A2, CYP2E1 or CYP3A4.

Preferably, the stress response protein encoded by the polynucleotide is a heat shock protein. More preferably, the heat shock protein is selected from the list consisting of Hsp70, Hsp90, Hsp110, calreticulin, gp96, grp170, Hsp27, Hsc70, Mycobacterium Hsp65, Legionella pneumophila Hsp60, Escherichia coli GroEL and GroES, and most preferably it is Hsp70. Alternatively, the polynucleotide encodes an inducer of stress protein expression that induces expression of a heat shock protein. Preferably it is selected from the list comprising heat shock factor-1 (HSF-1), heat shock factor-2 (HSF-2), heat shock factor-3 (HSF-3), interferon response factor-1 (IRF-1) or interferon response factor 2 (IRF-2).

In a second aspect, the invention provides a DNA vaccine comprising the above described products. By a ‘DNA vaccine’ is meant a product intended to elicit or enhance a therapeutic immune response comprising one or more polynucleotides encoding, and capable of expressing, proteins or peptides with immunogenic or adjuvant properties.

In a third aspect, the DNA vaccine comprises a polynucleotide encoding a toxin or prodrug-converting enzyme for enhancing an anti-tumour immune response. Preferably, the toxin or prodrug-converting enzyme is a nitroreductase capable of activating the prodrug CB1954. Alternatively, the toxin or prodrug-converting enzyme is a cytochrome P450. Preferably, the cytochrome P450 is selected from the list consisting of human CYP1A2, human CYP2E1, human CYP3A4, rodent CYP1A2, rodent CYP2E1 and rodent CYP3A4

Preferably, the toxin or prodrug-converting enzyme is capable of inducing necrotic cell death.

In a fourth aspect the invention provides a composition comprising a polynucleotide encoding a nitroreductase capable of activating the prodrug CB1954 and a polynucleotide encoding an immunostimulatory molecule, for use in enhancing an anti-tumour immune response. Included in the term ‘immunostimulatory’ are factors (such as IL-10 and TGF-β) that appear to function as inhibitors of immunosuppressive factors or effects.

Preferably, the immunostimulatory molecule is selected from the list consisting of GM-CSF, IL-1, IL-2, IL4, IL-6, IL-10, IL-12, IL-18, B7-2, TNFα, γ-IFN, MCP-1, MIP-2, RANTES, TGF-β, CD154 (CD40 ligand), CD134 ligand (OX40L), MHC Class I, MHC Class II, CD135 ligand (Flt3L), TNF-related apoptosis inducing receptor (TRAIL, Apo-2 ligand).

A particular embodiment of the invention is a vector encoding
a) a toxin or prodrug-converting enzyme and
b) a stress response protein,
   for use in enhancing an immune response.

Preferably, the immune response is an anti-tumour immune response.

It is also preferred that the stress response protein is a heat shock protein. More preferably the heat shock protein is selected from the list consisting of Hsp70, Hsp90, Hsp 110, calreticulin, gp96, grp170, Hsp27, Hsc70, Mycobacterium Hsp65, Legionella pneumophila Hsp60, Escherichia coli GroEL and GroES. Most preferably, it is Hsp70.

Preferably, the toxin or prodrug-converting enzyme is a nitroreductase capable of activating the prodrug CB1954. Most preferably, the vector is CTL102/mCMV-mHSP70, as shown in FIG. 1.

Alternatively, it is a cytochrome P450 enzyme, preferably of mammalian origin, more preferably of human origin. Most preferably it is human CYP1A2. Alternatively, it is human CYP2E1 or CYP3A4.

In an alternative preferred embodiment, the encoded enzyme is a rodent cytochrome P450, preferably from mouse. Most preferably, it is mouse CYP1A2, CYP2E1 or CYP3A4.

It is further preferred that any of the above described vectors have one or both of the polynucleotide sequences encoding of the toxin or prodrug-converting enzyme on the one hand, and the stress response protein or inducer of stress protein expression on the other, operably linked to one or more promoters providing tumour-selective expression.

The term “operably linked” as used herein refers to a cis-linkage in which the gene is subject to expression under control of the promoter.

Preferred tumour-selective promoters include TRP-1, HER2, HER3, ERBB2, ERBB3, CEA, MUC-1, α-fetoprotein, prostate specific antigen (PSA), villin, pancreatic amylase, tyrosinase related peptide, tumour rejection antigen precursor and T-cell factor (TCF) responsive promoters. Preferably the promoter comprises one or more TCF-responsive elements.

TCFs are a family of transcription factors within the High Mobility Group (HMG) of DNA-binding proteins (Love et al., Nature, 376, 791-795, 1995). The family includes TCF-1, TCF-3 and TCF-4 which are described in van der Wetering et al, (EMBO J., 10, 123-132, 1991), EP-A-0 939 122 and Korinek et al. (Science, 275, 1784-1787, 1997). TCF-4 has been shown to be involved in tumorigenesis related to Wnt/Wingless signalling. TCF and LEF-1 (lymphoid enhancer factor-1) are considered to mediate a nuclear response to Wnt signals by interacting with β-catenin. Wnt signalling and other cellular events that increase the stability of β-catenin are considered to result in transcriptional activation of genes by LEF-1 and TCF proteins in association with β-catenin. In the absence of Wnt signalling, LEF-1/TCF proteins repress transcription in association with Groucho and CBP (CREB binding protein).

In the absence of Wnt signalling, β-catenin is found in two distinct multiprotein complexes. One complex, located at the plasma membrane, couples cadherins (calcium dependent adhesion molecules) with the actin cytoskeleton whereas the other complex (containing the proteins adenomatous polyposis coli protein (APC), axin and glycogen synthase kinase 3β (GSK3β)) targets β-catenin for degradation. Wnt signalling antagonises the APC-axin-GSK3β complex, resulting in an increase in the pool of free cytoplasmic β-catenin. The free cytoplasmic β-catenin can translocate to the nucleus where it binds LEF-1/TCF factors and activates Wnt target genes. The regulation of LEF-1/TCF transcription factors by Wnt and other signals is discussed in Eastman et al., (Current Opin. Cell Biology, 11, 233-240, 1999).

TCFs are known to recognise and bind TCF binding elements which have the nucleotide sequence CTTTGNN, wherein N indicates A or T (van der Wetering et al, supra).

TCF-responsive elements have been shown to provide highly tumour selective expression of operably linked genes, especially in colon and liver tumours (WO 01/64739)

Preferably, the vector is a viral vector, more preferably, an adenoviral vector A specially preferred embodiment is an adenoviral vector encoding and allowing expression of (a) a nitroreductase capable of activating the prodrug CB1954 and (b) hsp70 for use in enhancing an anti-tumour immune response. Most preferably it is CTL102/mCMV-mHSP70.

Alternatively, the viral vector is a retroviral vector, more preferably a lentiviral vector.

A further embodiment is host cell comprising any of the above described vectors.

Another aspect of the invention is a vaccine comprising the product or composition of matter, vector, or host cell described above.

Also provided is the product or composition of matter, vector, or host cell described above for use as a medicament or vaccine.

A further aspect of the invention is a pharmaceutical composition comprising any of the above described products, compositions of matter, DNA vaccines, vectors or host cells together with a pharmaceutically-acceptable diluent, buffer, adjuvant or excipient.

Also provided is the use of any of the above described products, compositions of matter, DNA vaccines, vectors or host cells for the manufacture of a medicament for the treatment of cancer or for the manufacture of a vaccine for the treatment of cancer.

The invention provides a method of enhancing an immune response, comprising administering a therapeutic amount of a product comprising a polynucleotide encoding a toxin or prodrug-converting and a polynucleotide encoding a heat shock protein or an inducer of heat shock protein expression. Preferably, the immune response is an anti-tumour immune response.

There is also provided a method of treating a human suffering from a form of cancer, comprising administering a therapeutic amount of a product comprising a polynucleotide encoding a toxin or prodrug-converting enzyme and a polynucleotide encoding a heat shock protein or an inducer of heat shock protein expression.

In one preferred embodiment, the method comprises administering a therapeutic amount of a product comprising a polynucleotide encoding a nitroreductase capable of activating the prodrug CB1954 and a polynucleotide encoding a heat shock protein, allowing a period of time during which the product enters tumour cells and the encoded nitroreductase and heat shock protein are expressed, and administering a therapeutic amount of CB1954.

Alternatively, the method involves administering a therapeutic amount of a product comprising a polynucleotide encoding a cytochrome P450 and a polynucleotide encoding a heat shock protein, allowing a period of time during which the product enters tumour cells and the encoded cytochrome P450 and heat shock protein are expressed, and administering a therapeutic amount of a prodrug. Preferably the prodrug is acetaminophen.

It is preferred that any of the above described methods that the heat shock protein is Hsp70.

In another aspect of the invention is provided a method of treating a human suffering from a form of cancer, comprising administering a therapeutic amount of a product comprising a polynucleotide encoding a heat shock protein, and a therapeutic amount of anti-cancer cytotoxic drug such that a therapeutic anti-tumour immune response is induced.

In a preferred embodiment, the anti-cancer cytotoxic drug is capable of inducing necrotic cell death in tumour cells,

In a final aspect of the invention is provided a method of eliciting an anti-tumour immune response comprising administering a therapeutic amount of a product comprising a polynucleotide encoding a nitroreductase capable of activating the prodrug CB1954, allowing a period of time during which the composition enters tumour cells and the encoded nitroreductase is expressed, and administering a therapeutic amount of CB1954.

The product, DNA vaccine, vector or host cell of the invention or the pharmaceutical composition may be administered via a route which includes systemic, intramuscular, subcutaneous, intradermal, intravenous, aerosol, oral (solid or liquid form), topical, ocular, as a suppository, intraperitoneal and/or intrathecal and local direct injection.

The exact dosage regime will, of course, need to be determined by individual clinicians for individual patients and this, in turn, will be controlled by the exact nature of the protein expressed by the therapeutic gene and the type of tissue that is being targeted for treatment.

The dosage also will depend upon the disease indication and the route of administration.

The amount of nucleic acid construct or vector delivered for effective treatment according to the invention will preferably be in the range of between about 50 ng-1000 μg of vector DNA/kg body weight; and more preferably in the range of between about 1-100 μg vector DNA/kg.

Although it is preferred according to the invention to administer the nucleic acid construct, vector or host cell to a mammal for in vivo cell uptake, an ex vivo approach may be utilised whereby cells are removed from an animal, transduced with the nucleic acid construct or vector, and then re-implanted into the animal.

### BRIEF DESCRIPTION OF THE FIGURES

FIG. 1.

Structures of adenovirus serotype 5 (Ad5) E1 recombinant adenoviruses. All viruses are deleted for the viral regions E1 and E3 as indicated. The E1 deletion comprises nt 359-3524 and the E3 deletion nt 28,592-30,470 of the Ad5 viral genome. HCMV: human CMV (Cytomegalovirus) IE (immediate early) enhancer/promoter; NTR: E.coli initroreductase gene; IVSII: human β-globin intron II sequence; mCMV: mouse CMV IE enhancer/promoter; mHSP70: mouse Heat shock protein 70; SV40: SV40 late poly(A) signal; mGM-CSF: mouse granulocyte monocyte-colony stimulating factor.

FIG. 2.

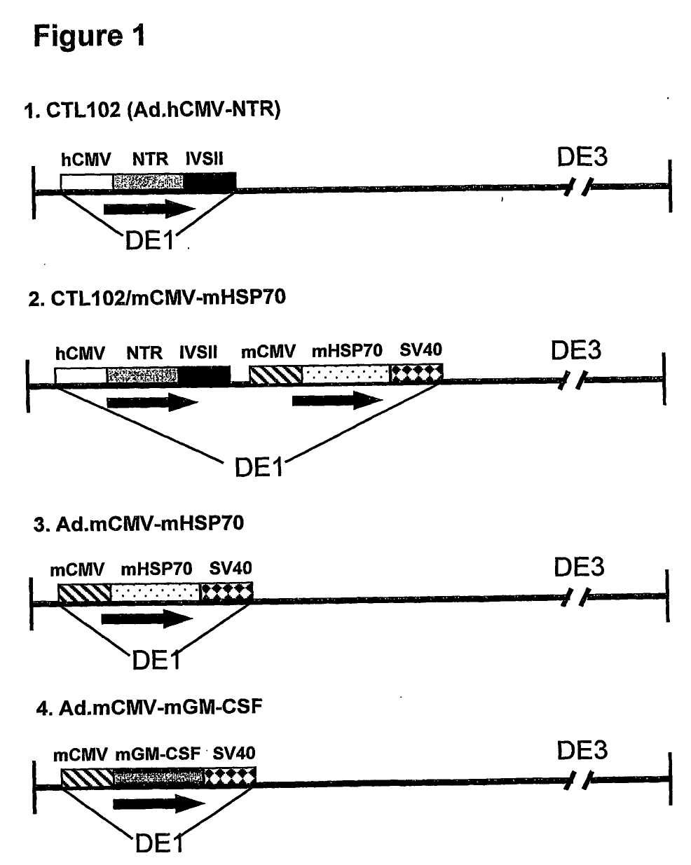

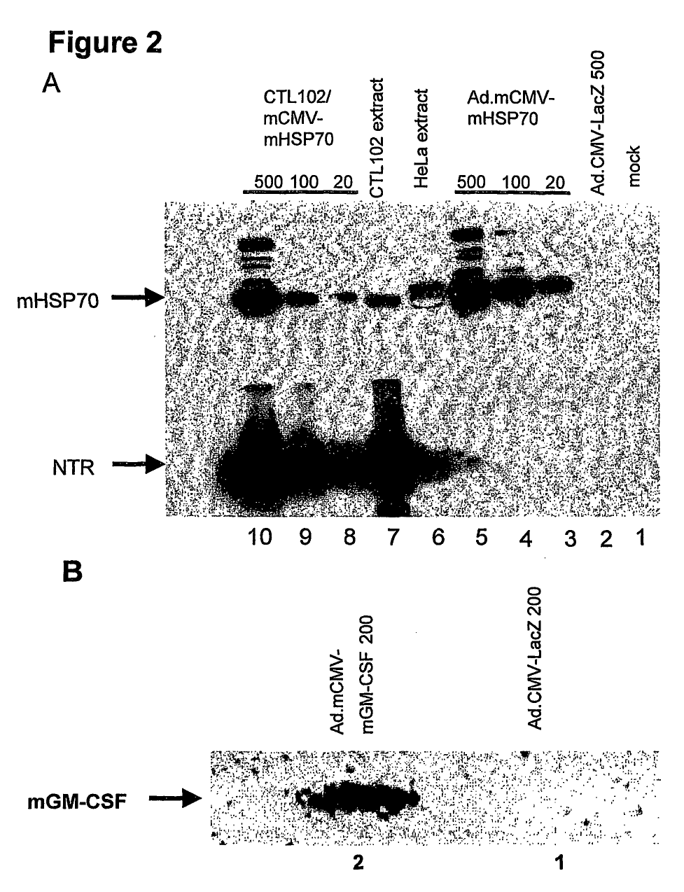

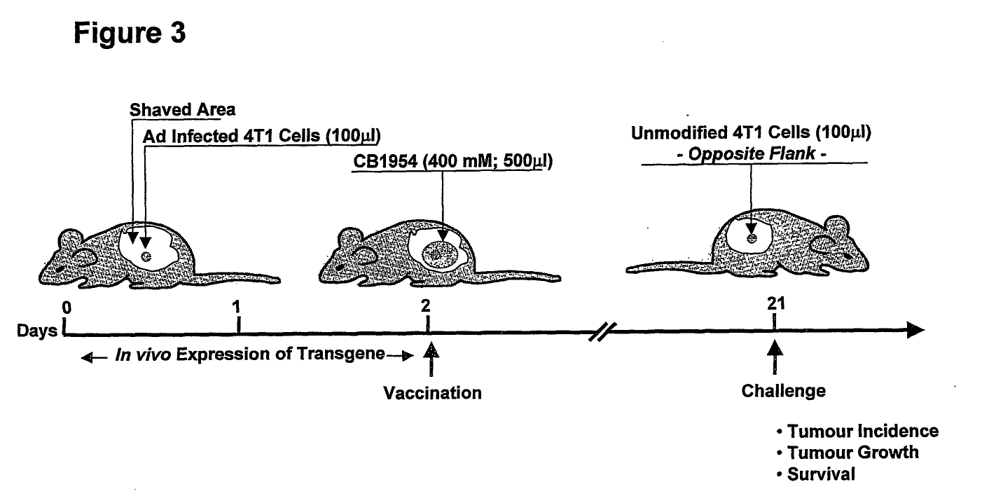

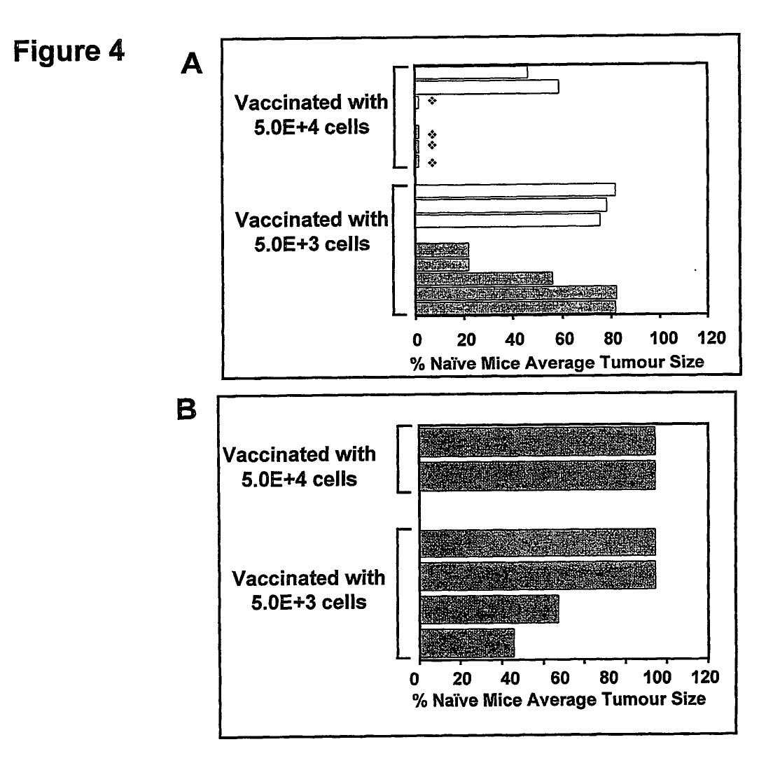

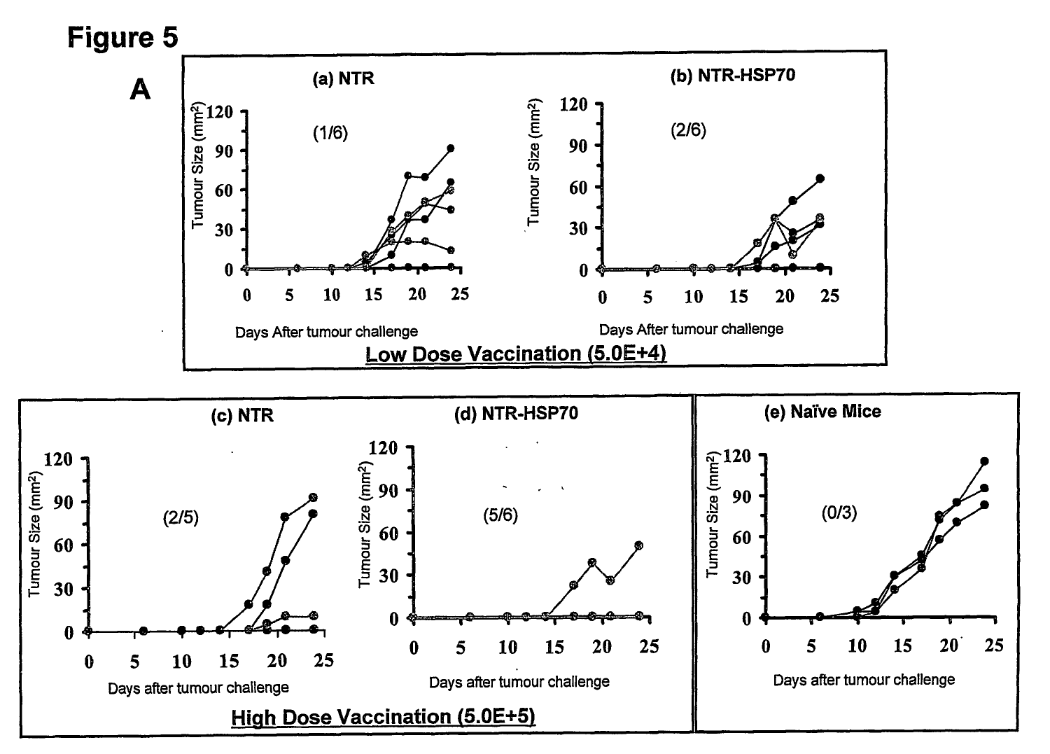

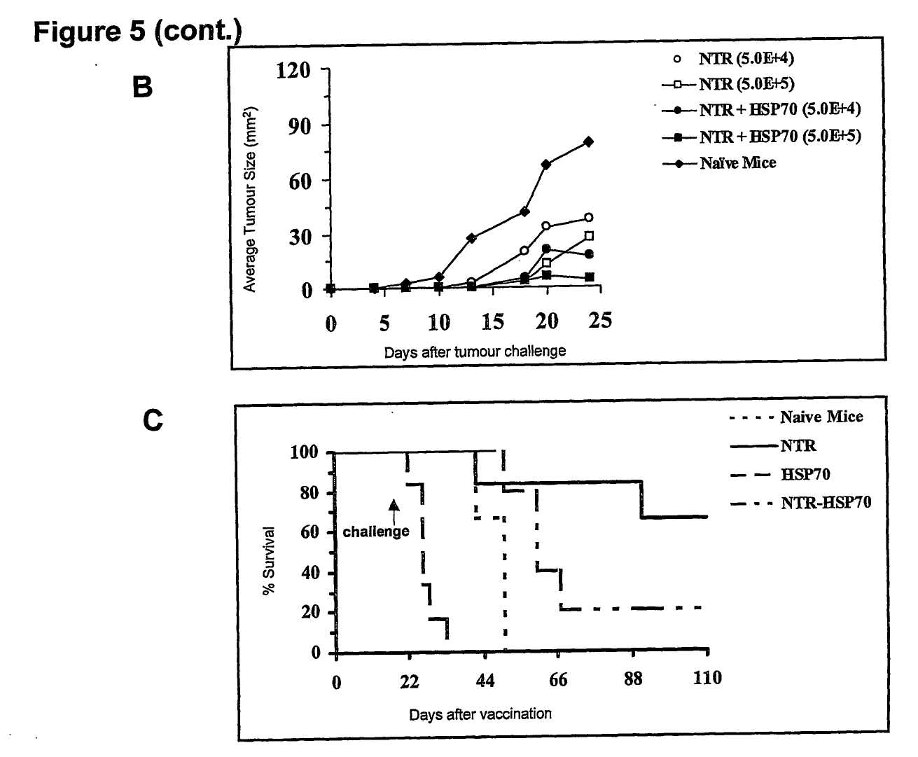

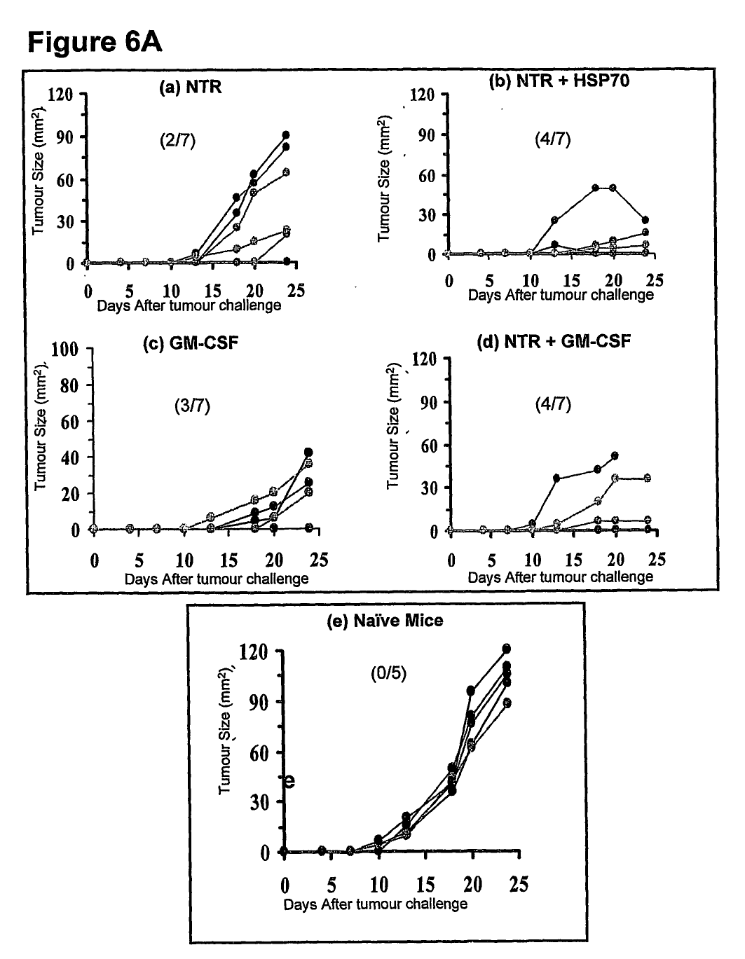

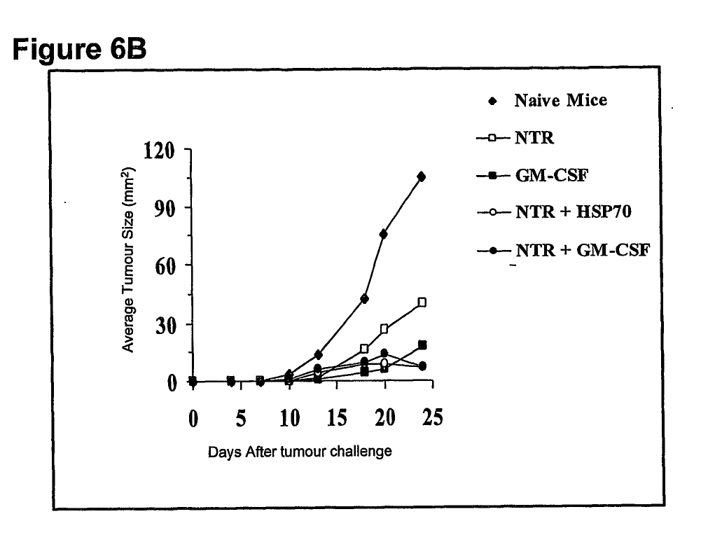

Western blot analysis of cellular lysates from Adenovirus vector infected cells analysing NTR, mHSP70 and mGM-CSF protein expression. (A) CMT93 cells (expressing no endogenous HSP70) were infected with the viruses Ad.mCMV-mHSP70 (lane 3-5) or CTL102/mCMV-mHSP70 (lane 8-10) with increasing MOI. CTL102 infected cell extract was used as positive control for NTR expression. Extract from Ad.CMV-LacZ (lane 2) infected cells was used as negative control. (B) HeLa cells were infected with Ad.mCMV-mGM-CSF (lane 2) or Ad.CMV-LacZ (lane 1; negative control). Whole cellular extracts were prepared 24 hours after infection. Denaturing SDS-PAGE was performed using 50 μg protein lysate and downstream processing was performed as described in Materials and methods. Biorad molecular weight standard was used to confirm the expected sizes of the expressed proteins.

FIG. 3.

Vaccination scheme.

FIG. 4.

A Protection of mice from challenge with 5.0×10³ parental 4T1 cells by a single vaccination with 5.0×10³ and 5.0×10⁴ Ad.hCMV-NTR-transduced (CTL102) 4T1 cells and in vivo CB1954 treatment. Mice were challenged either at 21 (open bars) or 80 (solid bars) days following vaccination. B Lack of protection from challenge with 5.0×10⁴ EJ6 cells, an irrelevant tumour cell line, at 21 days following vaccination with a single dose of 5.0×10³ or 5.0×10⁴ CTL102-transduced 4T1 cells and in vivo CB1954 treatment. Bars show the percentage of individual tumour size of challenge in treated mice in relation to the average tumour size of the control group of naïve mice at the time of compulsory sacrifice.

FIG. 5.

A Tumour size of challenge at 30 post-challenge in individual BALB/c mice vaccinated with (a) 5.0×10⁴ Ad.hCMV-NTR-transduced (CTL102) 4T1 cells and treated with CB1954, (b) 5.0×10⁴ Ad.hCMV-NTR/mCMV-mHSP70-transduced 4T1 cells and treated with CB1954, (c) 5.0×10⁵ CTL102-transduced 4T1 cells and treated with CB1954 and (d) 5.0×10⁵ Ad.hCMV-NTR/mCMV-mHSP70-transduced 4T1 cells and treated with CB1954. Twenty-one days following vaccination, all mice were challenged with 5.0×10³ parental 4T1 cells as well as a group of naïve mice (e). B Graph shows the average tumour size of treated groups. C Survival of mice vaccinated with 5.0×10⁵ CTL102-transduced 4T1 cells and treated with CB1954, 5.0×10⁵ Ad.mCMV-mHSP70-transduced 4T1 cells and treated with CB1954 and 5.0×10⁵ Ad.hCMV-NTR/mCMV-mHSP70-transduced 4T1 cells and treated with CB1954. Twenty-one days following vaccination, all mice were challenged with 5.0×10³ parental 4T1 cells as well as a group of naïve mice.

FIG. 6.

A Tumour size of challenge at 30 post-challenge in individual BALB/c mice vaccinated with (a) 5.0×10⁵ Ad.hCMV-NTR-transduced (CTL102) 4T1 cells and treated with CB1954, (b) 5.0×10⁵ CTL102+Ad.mCMV-mHSP70-transduced 4T1 cells and treated with CB1954, (c) 5.0×10⁵ Ad.mCMV-mGM-CSF-transduced 4T1 cells and treated with CB1954 and (d) 5.0×10⁵ CTL102+Ad.mCMV-mGM-CSF-transduced 4T1 cells and treated with CB1954. Twenty-one days following vaccination, all mice were challenged with 5.0×10³ parental 4T1 cells as well as a group of naïve mice (e). B Graph shows the average tumour size of treated groups.

### DETAILED DESCRIPTION OF THE INVENTION

The invention is described in detail by the use of the following examples. These are by way of illustration only and are not to be taken as limiting.

### EXAMPLES

### Example 1

Materials and Methods

Cell Culture

4T1, a mouse breast cancer cells were obtained from ATCC(CRL-2539). EJ-6-2-Bam-6a was obtained from ATCC(CRL-1888) and was generated by transfecting NIH/3T3 with DNA from the human EJ bladder carcinoma. PER.C6 cells (lit) were obtained from IntroGene (Leiden, The Netherlands). 911 cells were kindly provided by Prof. L. Young (CRC Institute for Cancer Studies, University of Birmingham, UK) and were maintained in DMEM containing 10% FCS, 10 mM MgCl₂ and antibiotics. 4T1 and EJ-6-2-Bam-6a were cultured as recommended by the supplier.

Plasmid Construction

pTX0374 was constructed by cloning a 1.6 kb BglII-BamHI fragment containing the human CMV promoter fused to the E. coli ntr gene (NTR: E. coli B/r nitroreductase gene amplified from genomic DNA) into pSW107. pRAJ 43 BP4 is a pUC19 plasmid containing the mouse GM-CSF cDNA and was kindly provided by Prof. L. Young (CRC Institute for Cancer Studies, University of Birmingham, UK. CET902 is a pUC19 plasmid containing the mouse CMV IE enhancer/promoter and the SV40 late poly(A) signal.

CET902/mCMV-mHSP70 contains an expression cassette for stress inducible mouse HSP70 protein and was constructed as described. First, the mouse full length (1.4 kb) CMV promoter was cloned into a pUC19-based vector. Second, the SV40 late poly(A) signal was cloned downstream from the promoter. Finally, the cDNA coding for mouse Hsp70 was cloned between the promoter and the poly(A) signal using a Smal site. A plasmid containing the cDNA for mouse HSP70 was kindly provided from Dr. R. Vile (Molecular Medicine Program, Mayo Clinic, 200 First Street SW, Rochester, Minn., USA) and was cut out as Nhel/BamHI fragment and blunted using T4 DNA polymerase.

CET902/mCMV-mGM-CSF was prepared by digesting pRAJ 43 BP4 with EcoRI and BamHI releasing a 465 bp fragment. The fragment was blunted and ligated into SmaI prepared CET902.

pPS1128 was kindly provided by Dr. P. Searle, CRC Institute of Cancer Studies, University of Birmingham. pPS1128 contains adenoviral sequences from the left hand ITR to nt 359 and from nt 3525 to 10,589 and is therefore an E1-deleted vector. pPS1128 was constructed by cloning a 917 bp fragment of the human beta-globin gene (BamHI site in exon2 to the EcoRI site in exon3) coupled to a 240 bp HincII-BamHI fragment containing the poly(A) addition and transcriptional termination signals of the human complement C2 gene into pBluescript (Stratagene). This plasmid was constructed in two stages. In the first, the left hand EcoRI site of pPS971 (Weedon et al, Int. J. Cancer, in press) was converted to a Swal site to create pPS115. In the second, the 350 bp Spe1-AMf/II fragment of pPS115 was replaced with a linker prepared by annealing the two oligonucleotides:

5′- CTAGTATCGATTGTTAATTAAGGGCGTGGCC -3′
and
5′- TTAAGGCCACGCCCTTAATTAACAATCGATA -3′.

pPS1022 was constructed from pPS972 by conversion of the right hand EcoRI site to a SwaI site.

pTX0375, the transfer vector used to generate CTL102, was constructed by cloning a SpeI fragment spanning the whole expression cassette (hCMV-NTR-IVSII-p(A)) from pTX0374 into SpeI digested pPS1128 and identification of a clone containing the cassette in the left to right orientation. pPS1128/mCMV-mHSP70 and pTXO375/mCMV-mHSP70 was constructed by transferring the mCMV-mHSP70-SV40 p(A) expression cassette from CET902/mHSP70 into blunted pPS1128 or into the blunted PacI site of pTX0375, respectively. The mCMV-mHSP70-SV40 p(A) expression cassette was prepared by digestion of CET902/mHSP70 plasmid with XmnI, AscI and BstZ171 and blunting with T4 DNA polymerase. Therefore the final cassette only contains about 500 bp of the mouse CMV promoter, this piece provides most of its activity. pPS118/mCMV-mGM-CSF was constructed by cloning the complete expression cassette (1.3 kb) prepared from CET902/mCMV-mGM-CSF (BstZ171 and AscI digested and blunted) into pPS1128.

The adenoviral “backbone” vector pPS160 was constructed by PacI linearisation of pPS128, ligation with a PacI-compatible adaptor (oligo1: 5′-TACATCTAGATMT-3′, oligo2: 5′-TTATCTAGATGTA-3′) containing an XbaI site followed by XbaI digestion to release a ca. 7 kb XbaI fragment containing Ad5 sequences 3524-10589. This was then cloned into XbaI linearised pPS1022 (Dr. Peter Searle) a pUC18-based plasmid containing Ad5 sequences from nt 10,589 to the right hand ITR but lacking nt 28,592 to 30,470 (E3 region).

Adenoviral Vector Construction

The recombinant viruses CTL102 (Ad.hCMV-NTR), CTL102/mCMV-mHSP70, Ad.mCMV-mHSP70 and Ad.mCMV-mGM-CSF were constructed by homologous recombination in Per.C6 cells. These cells were cotransfected with an equimolar mixture of pTX0375, pTXO3751mCMV-mHSP70, pPS1128/mCMV-HSP70 or pPS1128/mCMV-mGM-CSF, respectively, and pPS1160 into 90% confluent PER.C6 cells. The recombinant viruses were harvested about 7 days later by 3 freeze-thaw cycles in infection medium (DMEM, 1% FCS, 2 mM MgCl₂). By repeated infection/harvesting cycles the viruses were grown to large scale and then purified by standard CsCl density centrifugation, dialysed against excess of storage buffer (10 mM Tris pH 7.4, 140 mM NaCl, 5 mM KCl, 0.6 mM Na₂HPO₄, 0.9 mM CaCl₂, 0.5 mM MgCl₂ and 5% sucrose) and finally snap-frozen in liquid nitrogen and stored at −80° C. Particle concentrations were determined using the BCA Protein Assay Reagent (Perbio Science UK, LTD, Tattenhall, Cheshire, UK). Plaque forming units (p.f.u.) titres were determined by plaque assays on 911 cells.

4T1 in vitro Infection

4T1 cells were infected at a cell density of 1×10⁷/ml for about 2.5 hours in a humidified CO₂ incubator with the indicated p.f.u. per cell (MOI) in infection medium (normal medium but containing only 1% FCS). During infections cells were gently mixed every 30 minutes. Then, cells were pelleted (300×g, 5 minutes) and washed before being resuspended in PBS. In vaccinations experiments the MOI used was 200 to 300 and where combination viruses were used, the final dose of vector was kept constant to avoid a cytopathic dose-dependent effect.

Western Blot Analysis

3×10⁵ HeLa cells were infected with the indicated MOIs in infection medium by incubation for 2 hours at 37° C. in 5% CO₂. Cells were then fed with complete medium (10% FCS) and cultured for 24 hours. Cells were then washed in PBS and then a whole cell lysate was prepared by adding 200 μl RIPA buffer (10 mM Tris pH 8.0, 150 mM NaCl, 1.0% NP40, 0.1% SDS, 0.5% Na-Desoxycholat plus complete protease inhibitor cocktail, Roche) per 6-well. Cells were incubated for 10 min at RT and then centrifuged at 13,000 RPM at 4 C. The cleared lysate was transferred to a new tube and protein concentration determined using the Biorad DC Protein Assay kit (Hercules, Calif., USA). 30 μg of each lysate sample was resolved by 11% SDS-PAGE using High Rainbow protein size marker (Amersham Pharmacia, Piscataway, N.J., USA). Proteins were then transferred to a nitrocellulose membrane (Gelman Sciences, Ann Arbor, Mich., USA). The membrane was blocked in TBS (10 mM Tris pH 7.5, 150 mM NaCl)/0.1% Tween20/5% milk powder for 1 hour at RT. Primary antibodies were diluted in blocking buffer as follows: sheep anti-NTR (Polyclonal Antibodies, Dyfed, UK): 1:2000, mouse anti-HSP70 (SPA-810, Stressgen Biotechnologies, Victoria BC, Canada) 1:1000 and goat anti-GM-CSF (SantaCruz, Calif., USA; sc-1322): 1:1000. Membranes were incubated with primary antibody for 1 hour, RT and then extensively washed and then incubated for 30 min at RT with secondary antibodies in TBS/0.1% Tween20/0.5% milk powder as following: donkey anti-sheep-HRP (1:7,500; Sigma), anti-mouse-HRP (1:10,000; Sigma A-9917) and anti-goat-HRP (Sigma, A-5420). After extensive washings, enhanced chemiluminescence was carried out using Pierce “SuperSignal West Pico Chemiluminescence substrate” (Perbio Science UK Ltd., Tattenhall, UK) and analysed with Alpha Innotech Imager Model #2.3.1.

Mice

Female BALB/c mice (H-2^{d}, 6-8 weeks old) were obtained from Harlan (Oxion, UK) and were maintained in a temperature-controlled, light-cycled room with food and water ad libitum. Mice were allowed to rest for one week before any treatment. Care as well as all experimental procedures were conducted in full compliance with the UK Home Office regulations.

Murine Tumour Model

4T1 is a highly aggressive and metastatic cell line established from a mammary adenocarcinoma, which arose spontaneously in a BALB/cfC₃H mouse (Dexter et al, 1978). 4T1 cell line which expresses H-2^{d} class I but not class II molecules, is a nonimmunogenic tumour model, which does not stimulate a syngeneic antitumour response in vitro or in vivo. The minimal 100%-tumour-inducing dose in BALB/c mice is 5×10² cells. 4T1 cells produce aggressive solid tumours when injected subcutaneously into BALB/c mice that can spontaneously metastasise primarily to the lung while the primary tumour is growing in situ. The EJ-6-2-Bam-6a (Shih et al., 1981), a fibroblast cell line was used as a control tumour.

Vaccination Protocol

An indicated number of adenovirus-transduced 4T1 cells were injected s.c. into the right flank of each naïve mouse in a volume of 100 μl total (day 0). Different experimental groups were injected with cells transduced with single or different combinations of adenovirus containing the nitroreducatse and or co-stimulatory genes. After allowing for transgene expression to proceed in vivo till day 2, a time at which a maximal transgene expression was demonstrated to occur, a solution of 400 μM CB1954 was injected peritumourally in a total volume of 500 μl. Unless otherwise stated, on day 21 all mice were challenged with a second contralateral injection of 5.0×10³ parental 4T1 cells. This dose is 10-fold higher than the minimal lethal dose in naïve mice. A naïve group of mice was also injected with these cells at the same time. The induction of a long-term antitumoural immune response was demonstrated by challenging mice 80 days following vaccination. All groups of mice in any one experiment were challenged on the same day using the same preparation of cells. Animals were examined regularly for the appearance of tumours by palpation; whereafter tumours were measured in two perpendicular dimensions using a Vernier calliper 2-3 times/week. Tumour size was expressed as the product of the two diameters of individual tumours. Mice were culled for humane reasons when they exhibited signs of distress, the tumours becoming too necrotic or when tumours exceeded a size of 160 mm².

Results

CTL102/CB1954 killing of 4T1 can induce significant and prolonged protection against 4T1 cell challenge

To test the possibility that NTR/CB1954 tumour cell killing in situ may contribute to the generation of specific antitumour immune, vaccination experiments were conducted. In optimisation experiments to establish the optimum killing conditions where dose ranging of cell inoculation (5.0×10² to 5.0×10⁶ NTR-expressing 4T1 cells) and subsequently in vivo treatment with escalating CB1954 doses ranging from 100 to 400 μM were tried (data not shown), tumour-free mice were vaccinated. Mice that rejected the initial inoculation of tumour cells from two groups vaccinated with either 5.0×10³ or 5.0×10⁴ NTR-expressing 4T1 cells respectively were challenged with 5.0×10³ unmodified 4T1 cells on the opposite flank at 21 days following cell inoculation. Challenging mice 80 days following vaccination was also conducted to assess the induction of a long-term antitumoural immune response. All naïve mice developed progressively growing tumours by day 10 and reached compulsory sacrifice by day 25 post-challenge. A vaccine dose-dependent response of rejecting the challenge was observed in mice vaccinated with NTR/CB1954 killed cells irrespective of the time of challenge (FIG. 4A). Vaccination with a low dose of 5.0×10³ cells conferred a low level protection compared to naïve mice, while a higher vaccination dose (5.0×10⁴) enhanced considerably the immunogenecity of 4T1 cells and protected the majority of mice from challenge.

To assess the specificity of the protection seen with the NTR/CB1954 killing vaccine, mice vaccinated with either 5.0×10³ or 5.0×10⁴ NTR-expressing 4T1 cells were challenged with another syngeneic tumour, EJ6, a mouse fibrosarcoma. Immunisation with 4T1 cells failed to protect all mice against challenge with EJ6 cells (FIG. 4B).

Enhancement of Antitumour Immunity of NTR/CB1954 Killing by Expression of Murine HSP70 Costimulatory Molecules

To determine whether the effects of NTR/CB1954 killing in the vaccination model could be improved by expression of the costimulatory molecule HSP70, mice received a single immunisation consisting of 4T1 cells transduced with either the single recombinant virus carrying the NTR gene or the double recombinant virus carrying both NTR and HSP70 genes. Animals were injected peritumourally with CB1954 on day 2 and animals were then challenged with 5.0×10³ unmodified 4T1 cells on the opposite flank (see immunisation protocol). Two different vaccination doses were used, a low dose of 5.0×10⁴ and a high dose of 5.0×10⁵. All animals were challenged with 5.0×10³ unmodified 4T1 cells at 21 days post-cell inoculation and followed for tumour appearance, tumour growth and survival. The results suggest that NTR/CB1954 killing of cells expressing HSP70 is more potent in priming immune response than mice vaccinated with NTR/CB1954 killed cells alone (FIG. 5A). A higher number of vaccinating cells seem to be better in protecting mice against a subsequent challenge with 4T1 cells. Consequently, animals vaccinated with NTR-HSP70-expressing cells showed significant survival rate (FIG. 5C). By contrast animals receiving NTR-expressing cells show only a modest survival advantage with one mouse tumour-free surviving more than 3 months. Immunisation with cells expressing HSP70 alone and treated with CB1954 did not protect any mice against the emergence of tumours at the inoculation site where a progressively growing tumours developed that took approximately 30 days to reach the size of compulsory sacrifice and mice had to be sacrificed not long after the immunisation time of 21 days post-cell inoculation.

Enhancement of Antitumour Immunity of NTR/CB1954 Killing by Expression of mGM-CSF Costimulatory Molecules

To assess the effect of GM-CSF expression on the immune activating properties of NTR/CB1954 killing, 4T1 cells were co-transduced with NTR and GM-CSF carrying adenovirus vectors, injected into mice (5.0×10⁵) and treated in vivo with CB1954 (see immunisation protocol). Although, GM-CSF alone had a significant effect on rejection of challenge and was even superior to vaccination with the NTR/CB1954 killed cells, the presence of GM-CSF in the tumour environment at the time of NTR/CB1954 killing caused a much better protection against challenge with 57% of mice rejecting challenge at days 30 following challenge (FIG. 5A). All non-immunised, naïve mice had to be sacrificed because of tumour burden in less than a month following tumour challenge.

### REFERENCES CITED

1. Anderson W F (1998) Human gene therapy. Nature 392: (6679 Suppl): 25-30.
2. Bae M A, Pie J E and Song B J (2001). Acetaminophen induces apoptosis of C6 glioma cells by activating the c-Jun NH(2)-terminal protein kinase-related cell death pathway. Mol Pharmacol 60: 847-856.
3. Baler R, Dahl G and Voellmy R (1993). Activation of human heat shock genes is accompanied by oligomerization, modification and rapid translocation of heat shock transcription factor HSF1. Mol Cell Biol 13: 2486-2496.
4. Basu S, Binder R J, Suto R, Anderson K M and Srivastava P K (2000). Necrotic but not apoptotic cell death releases heat shock proteins, which deliver a partial maturation signal to dendritic cells and activate the NF-kappa B pathway. Int Immunol. 12: 1539-1546.
5. Basu S, Binder R J, Ramalingam T and Srivastava P K (2001). CD91 is a common receptor for heat shock proteins gp96, hsp90, hsp70 and calreticulin. Immunity 14: 303-313.
6. Boulares A H, Zoltoski A J, Stoica B A, Cuvillier O and Smulson M E (2002). Acetaminophen induces a caspase-dependent and Bcl-XL sensitive apoptosis in human hepatoma cells and lymphocytes. Pharmacol Toxicol 90:38-50.
7. Calos M P (1996). The potential of extrachromosomal replicating vectors for gene therapy. Trends in Genetics 12: 463-466).
8. Cao X, Ju D W, Tao Q, Wang J, Wan T, Wang B M, Zhang W and Hamada H (1998). Adenovirus-mediated GM-CSF gene and cytosine deaminase gene transfer followed by 5-fluorocytosine administration elicit more potent antitumor response in tumor-bearing mice. Gene Ther 5: 1130-1136.
9. Cotten M, Wagner E and Birnstiel ML (1992) Receptor-mediated transport of DNA into eukaryotic cells. Meth Enzymol 217: 618-644.
10. Dexter D L, Kowalski H M, Blazar BA, Fligiel Z. Vogel Ra and Heppner G H (1978). Heterogeneity of tumor cells from a single mouse mammary tumor. Cancer Res 38: 3174-81.
11. Emerson S G (1996). Ex vivo expansion of hematopoietic precursors, progenitors, and stem cells: the next generation of cellular therapeutics. Blood 87, 3082-3088
12. Fadok VA, Bratton DL, Rose DM, Pearson A, Ezekewitz R A and Henson PM (2000). A receptor for phosphatidylserine-specific clearance of apoptotic cells. Nature 405: 85-90.
13. Felzman T, Ramsey WJ and Blaese RM (1997). Characterization of the antitumor response generated by treatment of murine tumors with recombinant adenoviruses expressing HSVtk, IL-2, II-6 or B7-1. Gene Ther 4: 1322-1329.
14. Flotte TR, Afione SA, Conrad C, McGrath S A, Solow R. Oka H, Zeitlin P L, Guggino WB and Carter BJ (1993). Stable in vivo expression of the cystic fibrosis transmembrane conductance regulator with an adeno-associated virus vector. Proc Natl Acad Sci USA 90: 10613-10617
15. Friedlos F, Quinn J. Knox RJ and Roberts JJ (1992). The properties of total adducts and interstrand crosslinks in the DNA of cells treated with CB 1954. Exceptional frequency and stability of the crosslink. Biochem Pharmacol 43: 1249-1254.
16. Gallucci S, Lolkema M and Matzinger P (1999). Natural adjuvants: endogenous activators of dendritic cells. Nat Med 5: 1249-1255.
17. Gough MJ, Melcher AA, Ahmed A, Crittenden MR, Riddle DS, Linardakis E, Ruchatz AN, Emiliusen LM and Vile RG (2001). Macrophages orchestrate the immune response to tumor cell death. Cancer Research 61: 7240-7247.
18. Grove JI, Searle, PF, Weedon, SJ, Green NK, McNeish IA and Kerr DJ (1999). Virus-directed enzyme prodrug therapy using CB1954. Anti-Cancer Drug Design 14: 461-472.
19. Hengge UR, Chan EF, Foster RA, Walker PS and Vogel JC (1995) Cytokine gene expression in epidermis with biological effects following injection of naked DNA. Nature Genet 10: 161-166.
20. Hickman MA, Malone RW, Lehmann-Bruinsma K, Sih T R, Knoell D, Szoka FC, Walzem R, Carlson DM and Powell JS (1994). Gene expression following direct injection of DNA into liver. Human Gene Therapy 5: 1477-1483.
21. Hitt, MM, Addison CL and Graham, FL (1997) Human adenovirus vectors for gene transfer into mammalian cells. Advances in Pharmacology 40: 137-206.
22. Jones RK, Pope IM, Kinsella AR, Watson AJ and Christmas SE (2000). Combined suicide and granulocyte-macrophage colony-stimulating factor gene therapy induces complete tumor regression and generates antitumor immunity. Cancer Gene Ther 7: 1519-1528.
23. Keown WA, Campbell CR, Kucherlapati RS (1990). Methods for introducing DNA into mammalian cells. Methods Enzymol 185:527-37
24. Knox RJ, Friedlos F, Jarman M and Roberts JJ (1988). A new cytotoxic, DNA interstrand crosslinking agent, 5-(aziridin-1-yl)-4-hydroxylamino-2-nitrobenzamide, is formed from 5-(aziridin-1-yl)-2,4-dinitrobenzamide (CB1954) by a nitroreductase enzyme in Walker carcinoma cells. Biochem Pharmacol 37: 4661-4669.
25. Knox RJ, Friedlos F, Marchbank T and Roberts JJ (1991). Bioactivation of CB 1954: reaction of the active 4-hydroxylamino derivative with thioesters to form the ultimate DNA-DNA interstrand crosslinking species. Biochem Pharmacol 42: 1691-1697.
26. Kucherlapati and Skoultchi (1984) Crit. Rev. Biochem 16: 349-379.

27. Kuppner MC, Gastpar R, Gelwer S, Nossner E, Ochmann O, Scharneri A, Issels RD (2001). The role of heat shock protein (hsp70) in dendritic cell maturation: hsp70 induces the maturation of immature dendritic cells but reduces DC differentiation from monocyte precursors. Eur J Immunol 31:1602-1609.
28. Mack KD, Walzem R and Zeldis JB (1994). Cationic lipid enhances in vitro receptor-mediated transfection. Am J Med Sci 307: 138-143.
29. Mamane Y, Heylbroeck C, Genin P, Algarte M, Servant MJ, LePage C, DeLuca C, Kwon H, Lin R and Hiscott J (1999). Interferon regulatory factors: the next generation. Gene 237: 1-14.
30. Mathew A, Mathur SK, Jolly C, Fox SG, Kim S and Morimoto R1 (2001). Stress-specific activation and repression of heat shock factors 1 and 2. Mol Cell Biol 21: 7163-7171.
31. Matzinger P (1994). Tolerance, danger and the extended family. Annu Rev Immunol 12: 991-1045.
32. McNeish IA, Searle PF, Young LS and Kerr DJ (1997). Gene-directed enzyme prodrug therapy for cancer. Advanced Drug Delivery Reviews 26: 173-184.
33. Miller JL, Donahue RE, Sellers SE, Samulski RJ, Young NS and Nienhuis AW (1994). Recombinant adeno-associated virus (rAAV)-mediated expression of a human gamma-globin gene in human progenitor-derived erythroid cells. Proc Natl Acad Sci USA 91:10183-10187.
34. Mountain A (2000). Gene therapy: the first decade. Trends Biotechnol 18: 119-128.
35. Nakai A and Morimoto R1 (1993). Characterization of a novel chicken heat shock transcription factor, heat shock factor 3, suggests a new regulatory pathway.

36. Parsell DA and Lindquist S (1993). The function of heat-shock proteins in stress tolerance: degradation and reactivation of damaged proteins. Annu Rev Genet 27: 437-496.
37. Philip R, Brunette E, Kilinski L, Murugesh D, McNally M A, Ucar K, Rosenblatt J, Okarma TB and Lebkowski JS (1994). Efficient and sustained gene expression in primary T lymphocytes and primary and cultured tumor cells mediated by adeno-associated virus plasmid DNA complexed to cationic liposomes. Mol Cell Biol 14: 2411-2418
38. Plank C, Oberhauser B, Mechtler K, Koch C and Wagner E (1994). The influence of endosome-disruptive peptides on gene transfer using synthetic virus-like gene transfer systems. J Biol Chem 269: 12918-12924.
39. Russell DW, Miller AD and Alexander IE (1994). Adeno-associated virus vectors preferentially transduce cells in S phase. Proc Natl Acad Sci USA 91: 8915-8919.
40. Sakai Y, Kaneko S, Nakamoto Y, Kagaya T, Mukaida N and Kobayashi K (2001). Enhanced anti-tumor effects of herpes simplex virus thymidine kinase/ganciclovir system by codelivering monocyte chemoattractant protein-1 in hepatocellular carcinoma. Cancer gene Ther 8: 695-704.

41. Santodonato L, D'Agostino G, Santini SM, Carlei D, Musiani P, Modesti A, Signorelli P. Belardelli F and Ferrantini M (1997). Local and systemic antitumor response after combined therapy of mouse metastatic tumors with tumor cells expressing IFN-α and HSVtk: perspectives for the generation of cancer vaccines. Gene Ther 4: 1246-1255.
42. Sauter B. Albert ML, Francisco L, Larsson M, Somersan S and Bhardwaj N (2000). Consequences of cell death: exposure to necrotic tumor cells, but not primary tissue or apoptotic cells, induces the maturation of immunostimulatory dendritic cells. J Exp Med 191: 423-434.
43. Shih C, Padhy LC, Murray M and Weinberg R A (1981). Transforming genes of carcinomas and neuroblastomas introduced into mouse fibroblasts. Nature 290: 261-264.
44. Sikes ML, O'Malley BW Jr, Finegold MJ, Ledley FD (1994). In vivo gene transfer into rabbit thyroid follicular cells by direct DNA injection. Human Gene Therapy 5: 837-844.
45. Srivasatva (2002). Interaction of heat shock proteins with peptides and antigen presenting cells: chaperoning of the innate and adaptive immune responses. Annu Rev Immunol 20: 395-425.
46. Taniguchi T, Ogasawara K, Takaoka A and Tanaka N (2001). IRF family of transcription factors as regulators of host defense. Annu Rev Immunol 19: 623-655.
47. Trubetskoy VS, Torchilin VP, Kennel SJ, Huang L (1992). Use of N-terminal modified poly(L-lysine)-antibody conjugate as a carrier for targeted gene delivery in mouse lung endothelial cells. Bioconjugate Chem 3: 323-327
48. Ustav M and Stenlund A (1991). Transient replication of BPV-1 requires two viral polypeptides encoded by the E1 and E2 open reading frames. EMBO J. 10: 449-457.
49. Vile RG and Hart IR (1993). In vitro and in vivo targeting of gene expression to melanoma cells. Cancer Res 53: 962-967.
50. Walsh CE, Liu J M, Xiao X, Young N S, Nienhuis AW, Samulski RJ (1994). Regulated high level expression of a human gamma-globin gene introduced into erythroid cells by an adeno-associated virus vector. Proc Natl Acad Sci USA 89 :7257-7261.
51. Wilson JM, Grossman M, Wu CH, Chowdhury NR, Wu GY and Chowdhury JR (1992). Hepatocyte-directed gene transfer in vivo leads to transient improvement of hypercholesterolemia in low density lipoprotein receptor-deficient rabbits. J Biol Chem 267:963-967
52. Woff JA, Malone RW, Williams P. Chong W, Acsadi G, Jani A and Felgner PL (1990). Direct gene transfer into mouse muscle in vivo. Science 247: 1465-1468.
53. Wu GY and Wu CH (1988). Receptor-mediated gene delivery and expression in vivo. J Biol Chem 263:14621.

1. A product comprising a polynucleotide sequence encoding a toxin or prodrug-converting enzyme and a polynucleotide sequence encoding a stress response protein or an inducer of stress response protein expression.

2. The product of claim 1, for use in enhancing an immune response.

3. The product of either of claims 1 or 2 wherein the immune response enhanced is an anti-tumour response.

4. The product of any of claims 1 to 3 wherein the polynucleotide sequence encoding a toxin or prodrug-converting enzyme capable of inducing necrotic cell death and the polynucleotide sequence encoding a stress response protein or an inducer of stress response protein expression are both components of single polynucleotide molecule.

5. The product of any of claims 1 to 4 wherein the toxin or prodrug-converting enzyme is a nitroreductase capable of activating the prodrug CB1954.

6. The product of any of claims 1 to 4 wherein the toxin or prodrug-converting enzyme is a cytochrome P450.

7. The product of claim 5 wherein the cytochrome P450 is selected from the list consisting of human CYP1A2, human CYP2E1, human CYP3A4, rodent CYP1A2, rodent CYP2E1 and rodent CYP3A4.

8. The product of any of claims 1 to 7 wherein the stress response protein encoded or induced is a heat shock protein.

9. The product of claim 8 wherein the heat shock protein is selected from the list consisting of Hsp70, Hsp90, Hsp110, calreticulin, gp96, grp170, Hsp27, Hsc70, Mycobacterium Hsp65, Legionella pneumophila Hsp60, Escherichia coli GroEL and GroES.

10. The product of claim 9 wherein the heat shock protein is Hsp70.

11. A DNA vaccine comprising the product of any of claims 1-10.

12. A DNA vaccine comprising a polynucleotide encoding a toxin or prodrug-converting enzyme for enhancing an anti-tumour immune response.

13. The DNA vaccine of claim 12 wherein the toxin or prodrug-converting enzyme is a nitroreductase capable of activating the prodrug CB1954.

14. The DNA vaccine of claim 13 wherein the toxin or prodrug-converting enzyme is a cytochrome P450.

15. The DNA vaccine of claim 14 wherein the cytochrome P450 is selected from the list consisting of human CYP1A2, human CYP2E1, human CYP3A4, rodent CYP1A2, rodent CYP2E1 and rodent CYP3A4

16. A product comprising a polynucleotide encoding a nitroreductase capable of activating the prodrug CB1954 and a polynucleotide encoding an immunostimulatory molecule, for use in enhancing an anti-tumour immune response.

17. A product comprising a polynucleotide encoding a cytochrome P450 and a polynucleotide encoding an immunostimulatory molecule, for use in enhancing an anti-tumour immune response.

18. The product of either of claims 16 or 17 wherein the immunostimulatory molecule is selected from the list consisting of GM-CSF, IL-1, IL-2, IL-4, IL-6, IL-10, IL-12, IL-18, B7-2, TNFα, γ-IFN, MCP-1, MIP-2, RANTES, TGF-β, CD154, CD134 ligand, MHC Class I, MHC Class II, CD135 ligand and TRAIL.

19. A vector encoding and allowing expression of
a) a toxin or prodrug-converting enzyme and
b) a stress response protein,
for use in enhancing an immune response.

20. The vector of claim 19 wherein the immune response is an anti-tumour response.

21. The vector of either of claims 19 or 20 wherein the stress response protein is a heat shock protein.

22. The vector of claim 21 wherein the heat shock protein is selected from the list consisting of Hsp70, Hsp90, Hsp110, calreticulin, gp96, grp170, Hsp27, Hsc70, Mycobacterium Hsp65, Legionella pneumophila Hsp60, Escherichia coli GroEL and GroES.

23. The vector of claim 22 wherein the heat shock protein is hsp70.

24. The vector of any of claims 19 to 23 wherein the toxin or prodrug-converting enzyme is a nitroreductase capable of activating the prodrug CB1954.

25. The vector of any of claims 19 to 23 wherein the toxin or prodrug-converting enzyme is a cytochrome P450.

26. The vector of claim 25 wherein the cytochrome P450 is selected from the list consisting of human CYP1A2, human CYP2 μl, human CYP3A4, rodent CYP1A2, rodent CYP2E1 and rodent CYP3A4.

27. The vector of any of claims 19 to 26 wherein one or both of the polynucleotide sequences encoding of the toxin or prodrug-converting enzyme on the one hand, and the stress response protein or inducer of stress protein expression on the other, operably linked to one or more promoters providing tumour-selective expression.

28. The vector of claim 27 wherein the promoter comprises one or more TCF-responsive elements.

29. The vector of any of claims 19 to 29 wherein the vector is a viral vector.

30. The vector of claim 29 wherein the vector is an adenoviral vector.

31. The vector of claim 29 wherein the vector is a retroviral vector.

32. The vector of claim 31 wherein the vector is a lentiviral vector.

33. An adenoviral vector encoding and allowing expression of
a) a nitroreductase capable of activating the prodrug CB1954 and
b) hsp70
for use in enhancing an anti-tumour immune response.

34. A host cell comprising the vector of any of claims 19 to 33.

35. A vaccine comprising the product of any of claims 1 to 10, or 16 to 18, the vector of any of claims 19 to 33, or the host cell of claim 34.

36. The product of any of claims 1 to 10, or 16 to 18, the DNA vaccine of any of claims 10 to 15, the vector of any of claims 19 to 33, or the host cell of claim 34 for use as a medicament.

37. The product of any of claims 1 to 10, or 16 to 18, the vector of any of claims 19 to 33, or the host cell of claim 34 for use as a vaccine.

38. A pharmaceutical composition comprising composition of any of claims 1 to 10, or 16 to 18, the DNA vaccine of any of claims 10 to 15, the vector of any of claims 19 to 33, or the host cell of claim 34 together with a pharmaceutically-acceptable diluent, buffer, adjuvant or excipient.

39. Use of the product of any of claims 1 to 10, or 16 to 18, the DNA vaccine of any of claims 10 to 15, the vector of any of claims 19 to 33, or the host cell of claim 34 for the manufacture of a medicament for the treatment of cancer.

40. Use of the product of any of claims 1 to 10, or 16 to 18, the DNA vaccine of any of claims 10 to 15, the vector of any of claims 19 to 33, or the host cell of claim 23 for the manufacture of a vaccine for the treatment of cancer.

41. A method of enhancing an immune response, comprising administering a therapeutic amount of a product comprising a polynucleotide encoding a toxin or prodrug-converting enzyme and a polynucleotide encoding a heat shock protein or an inducer of heat shock protein expression.

42. The method of claim 41, wherein the immune response is an anti-tumour immune response.

43. A method of treating a human suffering from a form of cancer, comprising administering a therapeutic amount of a product comprising a polynucleotide encoding a toxin or prodrug-converting enzyme and a polynucleotide encoding a heat shock protein or an inducer of heat shock protein expression.

44. The method of any of claims 41 to 43, comprising
a) administering a therapeutic amount of a product comprising a polynucleotide encoding a nitroreductase capable of activating the prodrug CB1954 and a polynucleotide encoding a heat shock protein,
b) allowing a period of time during which the product enters tumour cells and the encoded nitroreductase and heat shock protein are expressed, and
c) administering a therapeutic amount of CB1954.

45. The method of any of claims 40 to 42, comprising
a) administering a therapeutic amount of a product comprising a polynucleotide encoding a cytochrome P450 and a polynucleotide encoding a heat shock protein,
b) allowing a period of time during which the product enters tumour cells and the encoded cytochrome P450 and heat shock protein are expressed, and
c) administering a therapeutic amount of a prodrug.

46. The method of claim 43 wherein the prodrug is acetaminophen

47. The method of any of claims 40 to 44 wherein the heat shock protein is Hsp70.

48. A method of treating a human suffering from a form of cancer, comprising administering a therapeutic amount of a product comprising a polynucleotide encoding a heat shock protein, and a therapeutic amount of anti-cancer cytotoxic drug, such that a therapeutic anti-tumour immune response is induced.

49. A method of eliciting an anti-tumour immune response comprising
a) administering a therapeutic amount of a product comprising a polynucleotide encoding a nitroreductase capable of activating the prodrug CB1954,
b) allowing a period of time during which the composition enters tumour cells and the encoded nitroreductase is expressed, and
c) administering a therapeutic amount of CB1954.
